(19) United States
(12) Patent Application Publication
(10) Pub. No.: US 2015/0273536 A1
(43) Pub. Date: Oct. 1, 2015

(54) MEDICAL INSTRUMENT CLEANING DEVICE AND RELATED METHODS OF USE

(71) Applicant: Ruhof Corporation, Mineola, NY (US)

(72) Inventors: Lee A. RUVINSKY, Mineola, NY (US); Bernard E. ESQUENET, Mineola, NY (US); Marc B. ESQUENET, Mineola, NY (US)

(73) Assignee: Ruhof Corporation, Mineola, NY (US)

(21) Appl. No.: 14/666,947

(22) Filed: Mar. 24, 2015

Related U.S. Application Data
(60) Provisional application No. 61/970,122, filed on Mar. 25, 2014.

(51) Int. Cl. B08B 3/10; A61B 19/00; A61L 2/18

(52) U.S. Cl. CPC B08B 3/10; A61L 2/18; A61B 19/34

(57) ABSTRACT

A method of cleaning a medical instrument using a cleaning device may include selecting a cleaning member for use with the cleaning device and attaching the cleaning member to the cleaning device. The method may also include submerging at least a portion of the cleaning device in a fluid actuating movement of the cleaning member. The method may further include contacting the cleaning member to the medical instrument.

### RELATED APPLICATIONS

This application claims the benefit of U.S. Provisional Application No. 61/970,122, filed Mar. 25, 2014, which is incorporated herein by reference in its entirety.

### FIELD OF THE DISCLOSURE

Embodiments of the present disclosure relate to the field of medical devices and, in particular, to methods and devices for cleaning medical instruments. More specifically, embodiments of the present disclosure are directed to a cleaning device and method of use that utilizes, e.g., a moveable cleaning member for cleaning medical instruments.

### BACKGROUND OF THE DISCLOSURE

Cleaning medical instruments can be a difficult task. For example, many instruments include sensitive and/or delicate portions or coatings. Others may include crevices or may have long, narrow components that are difficult to access. Complicating these issues is the fact that once cleaned, medical instruments may be handled by practitioners and used on other patients, and accordingly, medical facilities may require strict standards for the level of cleanliness achieved.

While some medical devices may be cleaned using autoclaves or chemicals, such sterilization techniques may not be practical for all medical instruments. Hand-scrubbing instruments may result in variations in pressure or torque applied to the instrument when scrubbing, which may result in instrument damage or inconsistent levels of cleanliness. Motorized cleaning equipment may include electronic components that are not be submersible in liquids, which may again complicate the cleaning process if used. The present disclosure proposes a novel device and method for cleaning medical instruments. Embodiments of the present disclosure may be able to overcome at least some of the issues presented by cleaning technology currently available in the medical field.

### SUMMARY OF THE INVENTION

In one aspect, the present disclosure is directed to a method of cleaning a medical instrument using a cleaning device. The method may include selecting a cleaning member for use with the cleaning device and attaching the cleaning member to the cleaning device. The method may also include submerging at least a portion of the cleaning device in a fluid and actuating movement of the cleaning member. The method may further include contacting the cleaning member to the medical instrument.

In some embodiments, movement of the cleaning member includes at least one of rotation, vibration, translation, oscillation, and reciprocation. In some embodiments, the method may further include agitating the liquid with the movement of the cleaning member. In some embodiments, the method may further include altering the movement of the cleaning member. In some embodiments, selection of the cleaning member may be based upon the medical instrument being cleaned.

In some embodiments, selection of the cleaning member may be predetermined based on a procedure for cleaning the medical instrument. In some embodiments, selection of the cleaning member may be based on the dirtiness of the medical instrument being cleaned. In some embodiments, selection of the cleaning member may be based on a material of the cleaning member and a material of the medical instrument.

In some embodiments, the method may further comprise replacing the cleaning member during the cleaning when it becomes worn out or damaged. In some embodiments, the method may further comprise submerging the entire cleaning device in the fluid. In some embodiments, the method may further comprise removing the cleaning device from the liquid and removing a battery from the cleaning device and replacing with a spare battery when the battery is low. In some embodiments, the method may further comprise recharging the battery removed from the cleaning device by plugging a charger cord into the battery.

In some embodiments, the method may further comprise actuating illumination of a light source of the cleaning device. In some embodiments, the method may further comprise configuring an articulation unit of the cleaning device to achieve a desired angle of deflection for an end effector of the cleaning device. In some embodiments, the cleaning member may be detachably connected to the end effector. In some embodiment, the cleaning member may include at least one of bristles, protrusions, and textured elements. In some embodiments, the cleaning device may be part of a kit that includes the cleaning device, a plurality of cleaning members, a spare battery, and a charger cord. In some embodiments, the kit may be specifically designed for cleaning the medical instrument. In some embodiments, wherein the cleaning members may be one of a bristled cleaning member, a textured cleaning member, a projection cleaning member, or a flexible cleaning member.

In another aspect, the present disclosure is direct to a method of cleaning a medical instrument. The method may include configuring an articulation unit of a cleaning device to achieve a desired angle of deflection for an end effector. The method may also include selecting a cleaning member to attach to the end effector and attaching the cleaning member to the end effector. The method may further include submerging at least a portion of the cleaning device in a fluid and actuating movement of the cleaning member. The method may also include actuating illumination of a light source on the end effector and contacting the cleaning member to the medical instrument.

Reference will now be made in detail to the exemplary embodiments of the present disclosure described below and illustrated in the accompanying drawings.

### BRIEF DESCRIPTION OF THE DRAWINGS

FIG. 1 is an illustration of a cleaning device, according to an exemplary embodiment;

FIGS. 2A, 2B, 2C, 2D, and 2E are illustrations of cleaning end effectors and cleaning members, according to an exemplary embodiments;

FIG. 3 is an illustration of a cleaning device and battery, according to an exemplary embodiment;

FIG. 4 is an illustration of a charger dock and power cable, according to an exemplary embodiment;

FIG. 5 is an illustration of a cleaning device, according to an exemplary embodiment;

FIG. 6 is an illustration of a cleaning device and end effector and cleaning member, according to an exemplary embodiment;

FIG. 7 is an illustration of a portion of a cleaning device and uncoupled end effector and cleaning member, according to an exemplary embodiment; and

FIG. 8 is an illustration of a kit that includes a cleaning device, a plurality of end effectors and cleaning members, a battery, and a charging cable, according to an exemplary embodiment.

FIG. 9 is a flow chart of a method of cleaning a medical instrument using a cleaning device, according to an exemplary embodiment.

The accompanying drawings, which are incorporated in and constitute a part of this specification, illustrate embodiments of the present disclosure and together with the description, serve to explain the principles of the present disclosure.

### DETAILED DESCRIPTION OF THE PREFERRED EMBODIMENTS

Reference will now be made in detail to the exemplary embodiments of the present disclosure described below and illustrated in the accompanying drawings.

While the present disclosure is described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the invention. Accordingly, the disclosure is not to be considered as limited by the foregoing or following descriptions. Other features and advantages and potential uses of the present disclosure will become apparent to someone skilled in the art from the following description of the disclosure, which refers to the accompanying drawings.

Prior to providing a detailed description of the embodiments disclosed herein, however, the following overview is provided to generally describe the contemplated embodiments. Further, although the embodiments disclosed herein are described in connection with devices for cleaning, e.g., medical instruments, those of ordinary skill in the art will understand that devices of the present disclosure may be suitable for other cleaning applications. Exemplary applications may include, e.g., industrial or household cleaning devices.

The disclosed embodiments relate to a cleaning device for medical instruments. The term “proximal,” as used herein, refers to a region closer to the body of a user when the device is being operated, and the term “distal” refers to a region further away from the user. The term “fluid” may include a state of matter or substance (liquid or gas), whose particles can move about freely and have no fixed shape, but rather conform to the shape of their containers. The term “medical instrument” may broadly refer to any medical device, tool, or item used in a medical facility, including, e.g., surgical instruments, catheters, incubators, stethoscopes, endoscopes, glidescopes, otoscopes, bed pans, stands, imaging equipment, probes, thermometers, neck braces, electrodes, splints, mouth blocks, or crutches. Further, the term “clean” or “cleaning,” as used herein, broadly refers to disinfecting, sterilizing, washing, scrubbing, rinsing, polishing, wiping, or otherwise removing debris or other substances from an instrument.

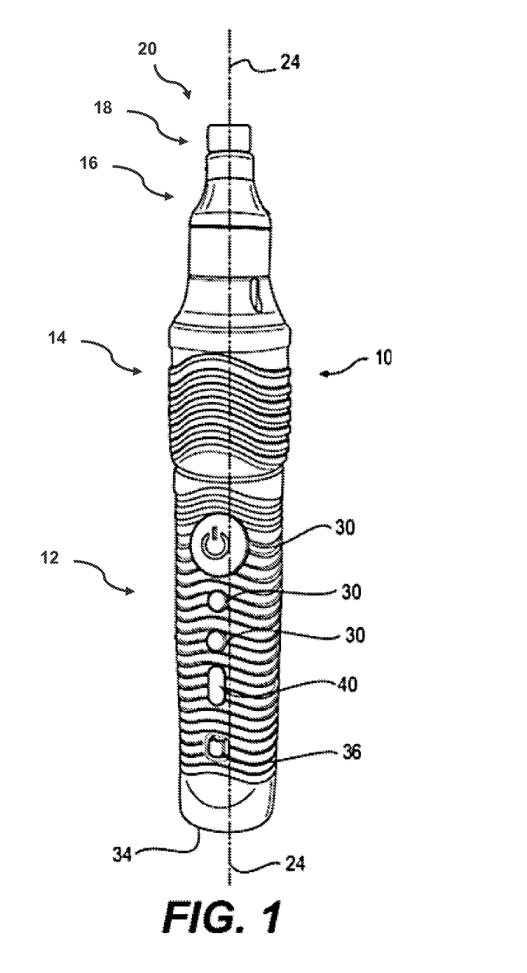

In one embodiment, as shown in FIG. 1, a cleaning device 10 for scrubbing medical instruments may include a base member 12, an articulation unit 14, and an end effector 16. End effector 16 may include a cleaning member 18 located at a distal end region 20 and one or more light sources 22. Articulation unit 14 joins end effector 16 to base member 12 and allows end effector 16 to move relative to base member 12. Joined by articulation unit 14, end effector 16 may extend from base member 12 so that base member 12 and end effector 16 align with each other along an axis 24 of cleaning device 10 when articulation unit 14 is in a neutral configuration. Articulation unit 14 may allow end effector 16 to extend at an angle to axis 24 of base member 12 when in a deflected configuration.

Cleaning device 10 may be used to clean one or more medical instruments. To clean an instrument, a user may grip base member 12 and contact a portion of end effector 16 to the instrument. Cleaning device 10 may be configured to withstand the application of force by a user against the instrument and to maintain a desired degree of articulation during the cleaning process. Each of the components, their functions, and the methods of using this cleaning device are described in further detail below.

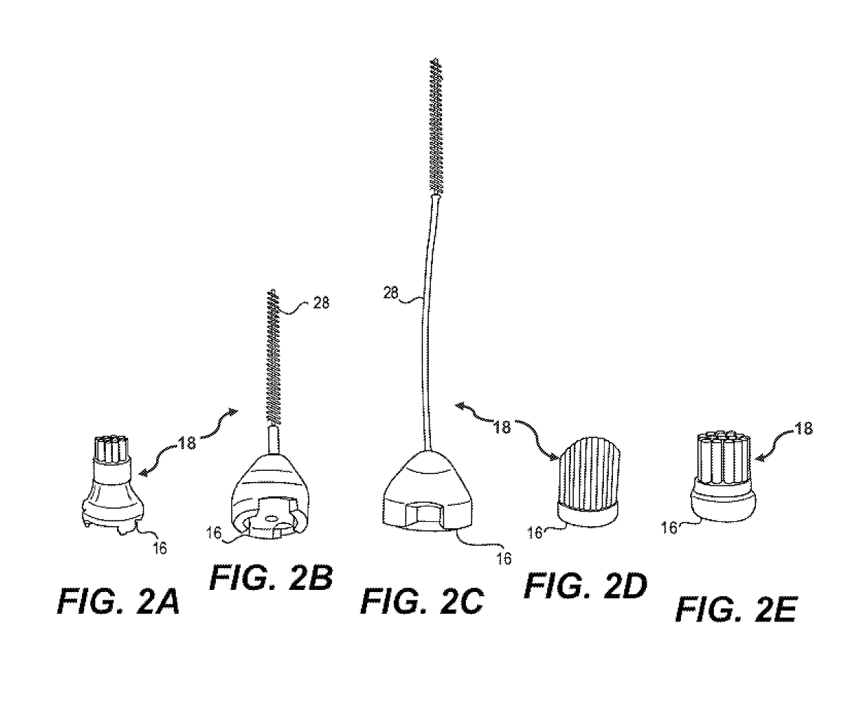

End effector 16 may include cleaning member 18 and be configured to contact a medical instrument in order to clean the instrument, for example, after using the instrument on a patient or in order to prepare the instrument for use. As shown in FIG. 1, cleaning member 18 may be located at distal end region 20 of end effector 16 and may extend from end effector 16 in line with axis 24 of cleaning device 10 when articulation unit 14 is in a neutral configuration. Cleaning member 18 may include one or more bristles, brushes, protrusions, textured faces (e.g., Velcro or sandpaper), picks, cloths, sponge, pads, foam, or any other suitable mechanism for cleaning. Cleaning member 18 may come in any suitable size and shape and may be formed of a wide variety of materials, e.g., natural or synthetic fibers, polymers, silicone, metal, cloth, rubber, etc. FIGS. 2A-2E show a variety of embodiments of end effector 16 and cleaning member 18.

In some embodiments, cleaning member 18 may include one or more bristles. The individual bristle(s) may have any suitable thickness or rigidity (or combinations thereof), and may be formed of any suitable material or combination of materials, e.g., nylon, plastic, rubber, silicone, metal, foam, etc. For example, the width of individual bristles may range from between approximately the width of a human hair (between 17-200 μm) to several millimeters thick. Bristle length may range from approximately a millimeter to several inches. Though stiffness varies depending on relative humidity and temperature, e.g., the tangent modulus, psi×10³ may range from approximately 20 to more than 2,500. The bristles may be spread out evenly or irregularly over an array, or clustered together regularly or irregularly, e.g., in a circular array, a rectangular array, a triangular array, or an array of any suitable shape. The array may be flat or mounded on a distal surface of cleaning member 18, and/or may extend along one or more side portions of a cleaning member, if cleaning member 18 has a more three-dimensional shape. The bristles may extend from the array coaxial with each other or may extend from the array at a plurality of different angles. The bristle array may be wider, narrower, or have an equal width relative to the rest of end effector 16, and the bristles may project out from end effector 16 any suitable distance, e.g., one millimeter or several inches. Additionally, the bristles may extend from a region of cleaning member 18 adjacent end effector 16, or cleaning member 18 may include one or more protrusions from which the bristles extend out from. The bristles may extend from cleaning member 18 in line with axis 24 of cleaning device 10 when articulation unit 14 is in the neutral position or may extend at an angle to axis 24.

In some embodiments, as shown in FIGS. 2B and 2C, cleaning member 18 may have an elongated protrusion 28 and may extend several inches or more than a foot from end effector 16. In such embodiments, protrusion 28 of cleaning member 18 may include one or more bristles at a distal region of protrusion 28 and/or on intermediate portions of the protrusion in order to clean medical instruments having hollow portions, crevices, cavities, or hard-to-reach locations, e.g., endoscopes. The bristles may extend from a distal-most surface of protrusion 28 or may extend from one or more sides of protrusion 28 instead of, or in addition to, the distal-most surface. The bristles may extend along one side of protrusion 28, along multiple sides of protrusion 28, along the entire length of protrusion 28, or along less than the entire length of protrusion 28. The bristles may be arranged evenly, irregularly, or clustered along the sides, or may be arranged in any suitable configuration, for example, forming one or more spirals around protrusion 28 or one or more rows or columns along protrusion 28. Protrusion 28 may be thin and generally cylindrical, or may be thicker, and protrusion 28 may have any suitable shape. The size and/or shape of protrusion 28 may vary along its length or may be constant. In some embodiments, protrusion 28 may be formed of braided or twisted metal wire, or may be a long, dowel-shaped rod. Additionally, protrusion 28 may be flexible, e.g., for accessing non-linear regions, or may be rigid.

In some embodiments, cleaning member 18 may include a portion of cloth, foam, cellulose, plastic, sandpaper, Velcro, or any other smooth or textured material or coating, or combinations thereof, suitable for wiping or abrading, e.g. This portion may take the shape of a relatively flat pad, may be more raised from end effector 16, or may be shaped as a protrusion. If cleaning member 18 includes a protrusion, the material may be located on a distal surface of the protrusion, or may cover one or more sides of the protrusion in addition to, or instead of, the distal surface. As with the bristle embodiment, cleaning member 18 may take any suitable size or shape.

In some embodiments, cleaning member 18 may include one or more elongated projections. For example, cleaning member 18 may include a single projection, such as a thin pick, extending out from end effector 16. This projection may include a sharpened, blunted, or shaped (e.g., angled) tip that may be used to clean crevices or small regions of medical instruments. Such projections may be formed of any suitable material, e.g., plastic, metal, rubber, etc. Additionally, the projections may be any suitable size or shape, e.g., more narrow, tooth-pick like projections, or wider, flat projections. The projections may have uniform cross-sections or cross-sections that vary along their length, e.g., they may taper or may resemble the taper of a screwdriver. The projections may or may not include one or more bristles. Again, like in the bristle embodiments above, these projections may extend out from end effector 16 on the scale of millimeters, several inches, or more than a foot, for example.

Cleaning member 18 may be permanently coupled to end effector 16 or may be detachable from end effector 16. For example, in some embodiments, cleaning member 18 may be capable of removal and replacement after being used, or end effector 16 may be capable of receiving different types of cleaning members. In some embodiments, different types of cleaning members may be included together in a kit, and a user may be able to select from among different types of cleaning members (e.g., different bristle members, a pick member, or textured polishing/abrading members) depending on the medical instrument or portion of the instrument to be cleaned. Accordingly, a user may interchangeably connect cleaning member 18 to a region of end effector 16.

In detachable embodiments, cleaning member 18 may connect to end effector 16 using any suitable mechanism. For example, one or more ridges, clips, or projections on a proximal portion of cleaning member 18 may mate with one or more cavities on end effector 16, or vice versa. Alternatively, cleaning member 18 may snap-fit, slide, screw, twist, clasp, friction-fit, lock-fit, magnetically couple, or otherwise attach to end effector 16 in any suitable manner. In some embodiments, a separate adapter may be used to couple a cleaning member to end effector 16.

Because cleaning device 10 may be used in more vigorous cleaning conditions, cleaning device 10 may be constructed in a rugged and durable manner so that whatever mechanism is used to connect cleaning member 18 to end effector 16, cleaning member 18 is secured well enough to remain connected to end effector 16 during use, while still allowing easy removal and attachment by the user. Additionally, for detachable embodiments, cleaning member 18 may be reusable or disposable. In some disposable embodiments, removing cleaning member 18 may alter cleaning member 18 so that it can no longer attach to end effector 16, for example, to prevent reuse.

In some exemplary embodiments, one or more portions of end effector 16 may be movable relative to base member 12 in order to facilitate cleaning of a medical instrument. For example, the portion of end effector 16 that supports cleaning member 18 may move relative to the rest of end effector 16 so that cleaning member 18 moves relative to the rest of cleaning device 10. In some embodiments, end effector 16 may be configured so that only cleaning member 18 may move relative to end effector 16. In other embodiments, a distal region of end effector 16 may move in addition to cleaning member 18, or the entire end effector 16 may move relative to base member 12. In still other embodiments, only a portion of cleaning member 18 may be moveable, while the rest of cleaning member 18 remains in place.

Exemplary types of movement may include, for example, rotating, vibrating, oscillating, translating, or reciprocating movement of cleaning member 18. Some embodiments may be capable of performing only one type of movement (e.g., rotating), while other embodiments may be capable of performing multiple types of movements. These movements may alternate (e.g., vibration and then rotation) or may occur simultaneously (e.g., vibration and rotation simultaneously). In some embodiments, the movements may be constant, may be capable of switching directions (e.g., rotating clockwise and then counterclockwise), may be capable of pulsing, or may be capable or changing speeds (e.g., two or three different speeds or more). Movement may be controlled by an actuator 30 located, for example, on base member 12. Using one or more actuators 30, a user may be able to select different movement types, different speeds, initiation and cessation of movement, etc, as discussed further below.

Cleaning member 18 of end effector 16 may allow a user to apply a more consistent and reproducible amount of torque and/or pressure to an instrument for cleaning. The movement and/or configuration of cleaning member 18 may allow a facility (e.g., a hospital, private practice, clinic, or industrial facility) to maintain certain standards of cleanliness. The inclusion of a dynamic cleaning member may mean that a user no longer needs to perform a scrubbing motion in order to clean a medical instrument. Elimination of the scrubbing motion may result in a more even application of pressure and/or torque throughout a single cleaning process and/or throughout cleaning processes generally. With cleaning member 18 supplying much of the cleaning motion and with control of this motion provided by actuator 30 on base member 12 (as will be described further below), the cleaning process may be more uniform. Additionally, if providers develop procedures regarding which types of cleaning members 18 should be used for cleaning which types of medical instruments, or even which portions of various instruments, standard, reproducible levels of cleanliness may be achieved. Further, reducing the amount of pressure and/or motion and/or controlling the type of cleaning members used on specific instruments may help to avoid damage of medical instruments during the cleaning process and may decrease user fatigue and increase ergonomics.

Moveable cleaning member 18 may also aid cleaning when cleaning device 10 is submerged in liquid. For example, movement of cleaning member 18 may agitate the liquid, which may aid in the cleaning process. Agitating the liquid may also help to achieve even temperature of the liquid and/or even distribution of a cleaning solvent mixed into the liquid, which may promote cleaning and/or protect against damage to the instrument. Movement of cleaning member 18 and/or agitation of liquid in which cleaning member 18 is submerged may create a more active cleaning process. Additionally, for any of the reasons listed above, a moveable cleaning member may decrease the time required to clean a medical instrument.

As shown in FIG. 1, end effector 16 may include one or more light sources. Light sources 22 may include, e.g., one or more light-emitting diodes (LED) or bulbs. Light source 22 may form a continuous periphery around a region of end effector 16, or one or more discrete light sources may be located on end effector 16. Light source 22 may be recessed within a surface of end effector 16, may lie flush with a surface of end effector 16, or may protrude from a surface of end effector 16, or different light sources 22 may be arranged in any suitable combination thereof. The one or more light sources 22 may be arranged on end effector 16 so as to provide directed lighting, e.g., to illuminate a cleaning surface for a user. For example, light sources 22 may be oriented so as to illuminate in a direction in line with cleaning member 18. In some embodiments, cleaning member 18 may extend from end effector 16 in line with axis 24 of cleaning device 10 when articulation unit 14 is in the neutral configuration, and light sources 22 may be oriented to shine a beam of light along axis 24. Accordingly, cleaning device 10 may extend linearly from base member 12 to cleaning member 18, and light sources 22 may provide illumination aimed in the direction of cleaning member 18. When articulation unit 14 is configured so that end effector 16 is deflected away at an angle, light source 22 and cleaning member 18 may deflect with end effector 16, and thus even when deflected, light source 22 may be oriented to shine a beam of light in line with cleaning member 18. In other embodiments, light sources 22 may be oriented so as to illuminate regions around end effector 16. Such arrangements may be useful, for example, when inserting cleaning member 18 into a hollow region or crevice. Light sources 22 may be configured to cast narrower beams of focused light or may cast wider beams of more diffuse light.

In some embodiments, a user may be able to select between multiple beam widths, multiple levels of brightness, and/or the number of light sources 22 lit at a given time. Additionally or alternatively, light sources 22 may be configured to emit different types of light (e.g., fluorescent, incandescent, UV, or colored lights), which may help a user to see certain types of residue that may be on an instrument during cleaning. For example, certain wavelengths of light may make certain dyes, bacteria, bodily fluids, etc., more noticeable, which may help a user determine whether all of the residue has been removed. In some embodiments, a user may have an option to select between types of light, or in other embodiments, the device may only include one type of light and the user can simply select between ‘on’ and ‘off’ or between brightness levels, beam widths, or manipulate which light sources are lit. A user may also be able to select between steady or pulsing light, for example. The type of light and/or control over the light may be controlled by an actuator 30, for example, located on base member 12, as will be discussed further below. Using one or more actuators 30, a user may be able to select different light types, different illumination levels, turn light sources 22 on and off, etc. In some embodiments, turning cleaning device 10 on and/or initiating movement of cleaning member 18 may automatically turn on light sources 22. In some embodiments, cleaning device 10 and end effector 16 may be configured to have no light sources 22.

End effector 16 and/or cleaning member 18 may further include any suitable coating. For example, either or both may include a waterproof coating, an antibacterial coating, a coating to protect the device from acidic or basic solutions, a stain-resistant coating, a corrosion-resistant coating, a scratch-resistant coating, etc.

Further, the entire end effector 16 or one or more portions of end effector 16 (and/or cleaning member 18, if attached) may be selectively coverable. For example, a cap may fit over a portion of end effector 16 when cleaning device 10 is not in use. This may allow cleaning member 18 or end effector to remain clean between uses or may provide protection while the device is stored and not in use. Such a cap may have any suitable shape and dimension for fitting over end effector 16 or a portion of end effector 16. The cap may be formed of any suitable substantially rigid material, e.g., metal, plastic, etc., in order to provide protection to end effector 16, though in some embodiments, the cap may be deformable in order to aid removal of the cap by a user. The cap may also include ridges or textured portions in order to facilitate removal and replacement by a user. The cap may couple to the device in any suitable manner, e.g., snap-fit, slide, screw, clasp, friction-fit, lock-fit, twist, or magnetically couple to end effector 16.

Additionally, in some embodiments, as shown in FIGS. 2A-2E, the entire end effector 16, and not only cleaning member 18, may be detachable from cleaning device 10. This may facilitate storage or cleaning, e.g., or may provide a user with end effectors 16 of different sizes or shapes to choose from. For example, some end effectors 16 may be wider or narrower to support different sized cleaning members 18 or to fit into narrower or larger spaces during cleaning. If end effector 16 is detachable, the assembly may be either reusable or disposable.

As shown in FIG. 1, base member 12 of cleaning device 10 may include an elongate portion configured to be gripped by the hand of a user. Base member 12 may have any suitable shape and may be dimensioned to facilitate one-handed or two-handed gripping. Base member 12 may have any suitable cross-sectional shape, including, but not limited to, oval, circular, rectangular, or irregular, and the cross-section may be uniform or may vary in size and/or shape along its length. For example, base member 12 may include one or more indentations or projections to facilitate positioning of the hand or certain fingers when gripping base member 12. Additionally, base member 12 may include textured portions, ridges, padding, friction-inducing coatings, or different materials, e.g., to reduce sliding of a user's hand and/or to increase comfort when using the device. Because the device is configured for cleaning, it is anticipated that a user may wear gloves when operating the device. Accordingly, in some embodiments, the shape and/or texturing of base member 12 may be designed so as to make base member 12 comfortable and easy to grip even when wearing hand protection.

Further, base member 12 may include one or more actuators 30 to control one or more portions of cleaning member 18, end effector 16 and/or articulation unit 14. For example, actuator(s) 30 may include a button, lever, switch, knob, dial, or any other suitable mechanism. Because a user may handle cleaning device 10 while wearing gloves, actuator(s) 30 may be sized and positioned to allow for easy activation even when wearing hand protection. In an exemplary embodiment, actuator 30 may include a single button to power the device on and off. Powering the device on may cause cleaning member 18 and/or portions of or the entire end effector 16 to move. Pressing the power button may also cause one or more light sources 22 to turn on. Pressing the same button multiple times may provide control over e.g., different movement speeds, different movement types, different brightness levels of light, or different types of light. In this way, a single actuator 30 may provide control over several different actions of end effector 16. Having one button control all aspects of end effector 16 may allow a user to easily manipulate cleaning device 10 with one hand while holding a medical instrument in the other hand during the cleaning process. In other embodiments, base member 12 may include more than one actuator 30, or more than one type of actuator 30, which may allow for separate control over different actions of end effector 16. For example, one button may control light sources 22 while a separate button controls movement of cleaning member 18.

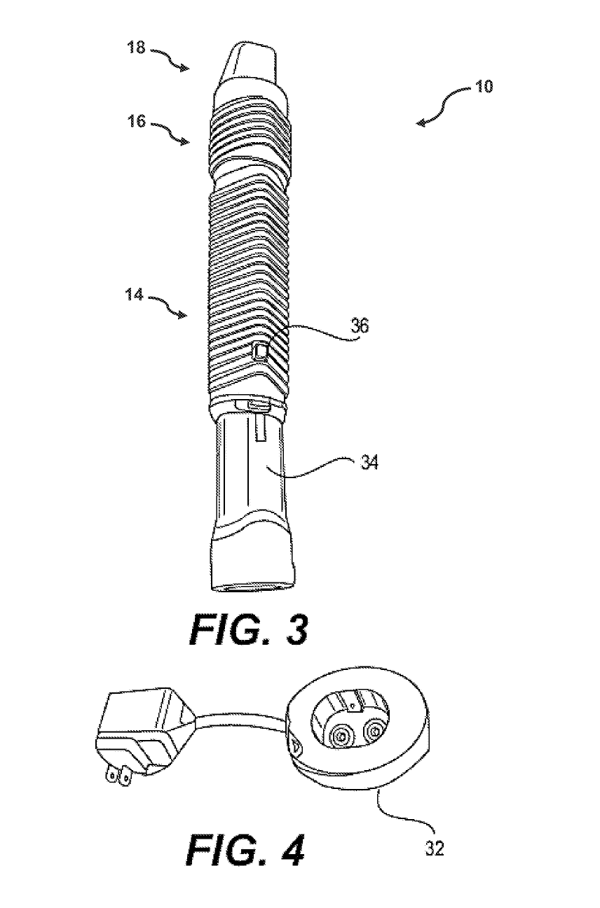

Base member 12 may also contain a suitable motor for causing movement of end effector 16. The motor may be powered by any suitable device, for example, a battery including a replaceable battery or a rechargeable battery. In embodiments including a rechargeable battery, base member 12 may include a portion configured to connect to a charging dock or a port for receiving a charger cable. For example, a portion of base member 12 may be configured to connect to, or be received by, the charging dock, and charging dock may plug into a power source. In rechargeable embodiments, base member 12 and/or the charging dock may include one or more indicator lights for indicating the charging status of the battery of cleaning device 10. In embodiments for use with one or more removable batteries (whether replaceable or rechargeable), base member 12 may include a removable portion for separating battery from base member 12, as shown in FIG. 3, or a removable panel for accessing an in internal battery storage cavity. In such embodiments, base member 12 may also include one or more tabs 36, buttons, levers, sliding, twisting, or screwing portions, or any suitable mechanism for replaceably removing a panel or portion of base member 12. In some embodiments, as shown in FIG. 4, a rechargeable battery 34 may separate from base member 12 and, once removed, may separately interface with a charging dock 32 for recharging battery 34, as shown in FIG. 4. In other embodiments, base member 12 may include a cord and may be configured to plug into an outlet in addition to, or instead of, including battery 34.

Base member 12 may also include an indicator light 40. Indicator light 40 may simply turn on and off, may be constant, may blink at a constant rate or at a variable rate, may change color, or may emit light in any other manner in order to indicate a status of the device to the user. For example, indicator light 40 in base member 12 may turn on when cleaning device 10 is activated and may turn off when cleaning device 10 is not activated. Indicator light 40 may turn on or may blink or change color to indicate low battery or to indicate that battery 34 is charging. Indicator light 40 may also be used to indicate the speed of motion selected, the type of motion selected, the type of light source 22 illuminated, or any other suitable setting, status, or parameter of the device.

Base member 12 may also include other components, for example a hook to facilitate storage or a loop to allow a user to secure cleaning device 10 around a wrist when operating cleaning device 10.

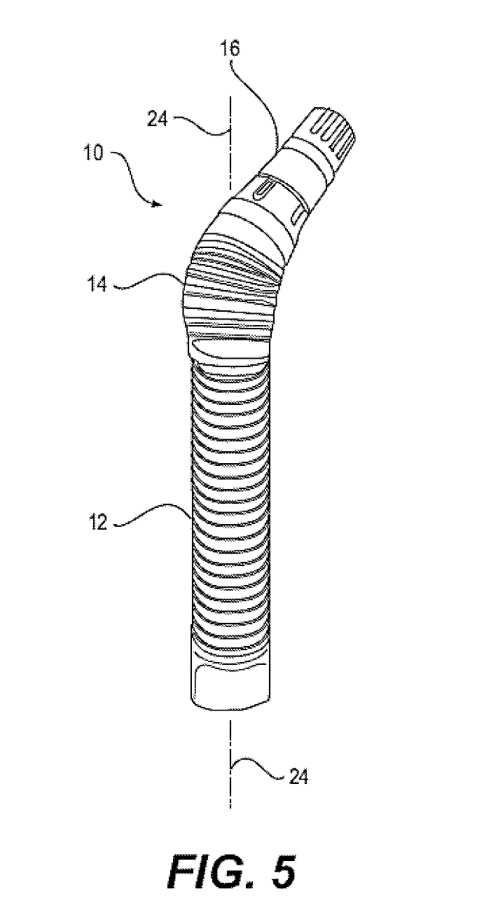

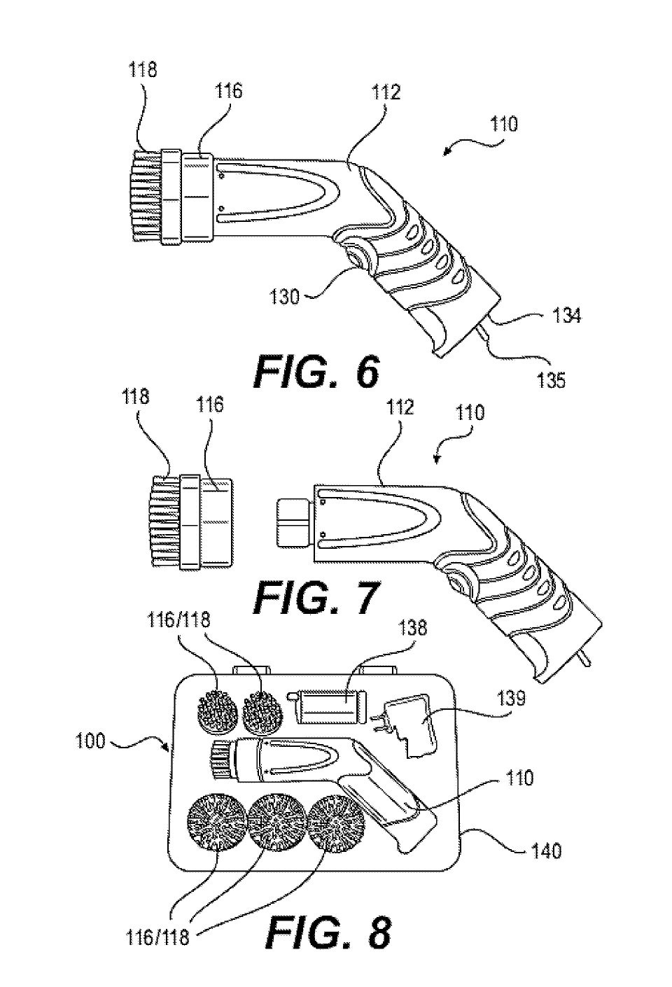

Articulation unit 14 may be located between base member 12 and end effector 16. As discussed previously, base member 12 and end effector 16 may extend in line with each other along axis 24 when articulation unit 14 is in a neutral configuration, and articulation unit 14 may allow end effector 16 to deflect at an angle to axis 24 when articulation unit 14 is in a deflected configuration, as shown in FIG. 5. Accordingly, base member 12 may allow end effector 16 to deflect at an angle, or at a range of optional angles, away from axis 24. Articulation unit 14 may allow for deflection of end effector 16 at a pre-set number of articulation angles or may allow for a continuous range of deflection at any angle up to a maximum angle, e.g., 90 degrees, from axis 24. Additionally, in order to allow a user to apply force to a medical instrument when using cleaning device 10, as may occur, e.g., when cleaning an instrument, articulation unit 14 may lock in place to prevent additional deflection or movement of end effector 16 during cleaning once the desired angle of articulation is selected by the user.

In some embodiments, cleaning device 10 may be configured to permit deflection in multiple directions. In such embodiments, end effector 16 may be able to deflect towards the user or away from the user, for example, and/or to one or more sides. In other embodiments, cleaning device 10 may be capable of articulating only in one direction, e.g., either towards the user or away from the user, or to only one side. The directions towards and away from the user as used herein are purely exemplary, and it should be understood that end effector 16 may be capable of articulating in any direction away from axis 24, whether exclusively in that single direction or in multiple alternative directions.

Articulation unit 14 may include any suitable mechanism for enabling deflection from axis 24. For example, any suitable joint may be used to moveably connect end effector 16 to base member 12, including, e.g., a pivot joint, saddle joint, revolute joint, knuckle joint, hinge joint, or ball-and-socket joint. In one exemplary embodiment, the articulation unit may include two arms extending distally from base member 12. Each arm may include an opening. A moveable, cup-shaped portion of articulation unit 14 may rest between the two arms, so that projections extending out from the sides of the cup-shaped portion fit through or otherwise engage the arm openings so that the cup-shaped portion is held in place between the arms. This configuration may allow the cup-shaped portion to pivot freely back and forth, and a proximal surface of the cup-shaped portion located between the bases of the two arms may be rounded to facilitate rocking movement of the cup. End effector 16 may connect to a distal region of the cup-shaped portion, causing end effector 16 to move when the cup-shaped portion moves and thus pivot relative to base member 12. When the cup-shaped portion is upright, end effector 16 may lie along axis 24 of the device in the neutral configuration. When the cup-shaped portion pivots off axis 24, end effector 16 may correspondingly deflect off axis 24. Although the term “cup-shaped portion” is used in this example to describe a component of the articulation joint, one of ordinary skill in the art will understand that the cup-shaped portion can in fact have any suitable shape and may not be hollow.

Articulation unit 14 may further include a locking mechanism to limit the range of motion of the cup-shaped portion and/or lock the cup-shaped portion in place once an angle of articulation has been selected by a user. In one exemplary embodiment, a ratchet mechanism may be included to limit the range of motion of the cup-shaped portion, for example, fixing the number and degree of available deflection angles, and locking the cup-shaped portion in place once an angle is selected. In one exemplary embodiment, the proximal surface of the cup-shaped portion may include a column of parallel ridges extending along the axis of movement of the cup. A surface of articulation unit 14 between the arms of the articulation joint near the proximal surface of the cup-shaped portion may include a pawl biased distally towards the proximal surface of the cup-shaped portion. Biasing of the pawl may be achieved using a rigid material capable of flexing under the application of a certain amount of force, or may be achieved using a spring or other biasing mechanism pushing the pawl distally towards the proximal surface of the cup. Accordingly, the pawl may be biased to engage the ridges on the proximal surface of the cup, preventing movement of the cup-shaped portion unless a certain amount of force is applied to the cup-shaped portion in order overcome the bias, at which point the pawl may be pushed out of place. Once pushed out of place, the pawl may glide past one or more ridges (depending on the amount of force applied by the user) until the user ceases to apply force, at which time the pawl will come to rest biased against the next ridge. Thus, by applying a certain amount of force, incremental deflection of end effector 16 can be achieved using a ratchet mechanism. In this manner, force may be applied to the articulation joint (or a region distal to the cup-shaped portion of the articulation joint) in order to deflect end effector 16 away from axis 24 at a distance proportional to one ridge-length. Applying additional force may allow for additional deflection of end effector 16 away from axis 24 proportional to the number of ridges that the force has caused the pawl to slide past.

In other embodiments, locking of articulation unit 14 may be controlled by one or more actuators 30 included in base member 12. For example, a user may deflect end effector 16 to a desired angle and then actuate actuator 30 to lock articulation unit 14 and maintain end effector 16 in that location. Alternatively, a user may actuate actuator 30 in order to deflect end effector 16, and end effector 16 may once again be locked when actuator 30 is released. In some embodiments, one actuator may allow for movement of end effector 16, and one actuator 30 may allow for locking of end effector 16. Thus, a separate actuator 30 or the same actuator 30 may allow a user to unlock articulation unit 14 to reposition the angle of deflection.

As discussed above, any suitable articulation joint and locking mechanism may be used to achieve incremental or continuous deflection of end effector 16 from axis 24. For example, articulation unit 14 may include one or more ratchet mechanisms, gears, pivoting members, or any other suitable mechanism, so long as end effector 16 is capable of easy articulation by a user while remaining resilient enough to allow for the forces applied to cleaning device 10 during cleaning without further articulation. For example, suitable joints and locking mechanisms may be capable of resisting forces from approximately 1 psi to over 50 psi, depending on the embodiments, during the cleaning process.

Additionally, articulation unit 14 may be configured to allow wires to pass through the unit from base member 12 to end effector 16. For example, articulation unit 14 may include one or more openings, slots, or channels through which wires can extend to transmit power and/or signals between components of base member 12 (e.g. a battery) and components of end effector 16 to perform an action (e.g., moving cleaning member or light source). Accordingly, the wires may be capable of passing through articulation unit 14 without becoming kinked, caught, or otherwise damaged by deflection and locking of articulation unit 14.

Further, articulation unit 14 may not be visible to a user. For example, articulation unit 14 may be covered by a flexible and/or deformable material, such as rubber, silicone, or a suitable polymer, to allow for deflection of articulation unit 14 while hiding articulation unit 14 from the view of a user and/or protecting the unit components.

Base member 12, articulation unit 14, and end effector 16 may be formed of any suitable material or combinations of materials, for example, metals, polymers, rubber, silicone, etc. Any portion of cleaning device 10 may include one or more coatings, e.g., antibacterial, waterproof, stain-resistant, corrosion-resistant, or scratch-resistant coatings. Further, cleaning device 10 may be sealed, for example, at seams or joints, to provide sufficient waterproofing capable of preventing seepage of liquid into cleaning device 10 even when cleaning device 10 is submerged in liquid during cleaning to allow for activation and use when completely submerged.

In another embodiment, a cleaning device may be configured such that base member 12 connects directly to end effector 16, thereby eliminating articulation unit 14. For example, FIG. 6 shows a cleaning device 110, according to another embodiment. Cleaning device 110 may have all the same or similar components to cleaning device 10, as described herein. For example, cleaning device 110 may include a base member 112, end effector 116, cleaning member 118, actuator 130, and rechargeable battery 134. Unlike cleaning device 10, end effector 116 of cleaning device 110 may couple directly to base member 112. As shown in FIG. 6, base member 112 may include a proximal grip portion which is at an angle to the distal portion at which end effector 116 may be connected. For cleaning device 110, the angle between the proximal portion and distal portion may be permanently fixed (i.e., not adjustable).

As shown in FIG. 6, cleaning device 110 like cleaning device 10 may be configured such that end effector 116 and cleaning member 118 may be releasably coupled to base member 112. Battery 134 of cleaning device 110 may be removable from the proximal end of base member 112. A ring 135 may be connected to the end of battery 134 thereby enabling a user to pull on ring 135 in order to remove battery 134. Battery 134 may have a plug configured to directly receive a charging cord, thereby eliminating the need for a charging dock.

Battery 134 may also have an o-ring configured to seal the connection between base member 112 and battery 134. The connection between base member 112 and battery 134 may be liquid tight, thereby enabling cleaning device 110 to be entirely submerged in liquid when cleaning a medical instrument like cleaning device 10.

Cleaning device 110 may be provided as part of a kit 100 for cleaning medical instruments. Kit 100 may be specifically designed for cleaning one or more kinds of medical instruments. As shown in FIG. 8, kit 100 may include a cleaning device 110, a spare battery 138, a charging cable 139, and a plurality of end effectors 116 having cleaning members 118 attached. The components of kit 100 may be provided in a case 140. End effectors 116 and cleaning members 118 provided in kit 100 may be different sizes. For example, each end effector 116 and cleaning member 118 provided may be of a different size. Alternatively, multiple end effectors 116 and cleaning members 118 of the same size may be provided. For example, as shown in FIG. 8, three smaller diameter end effectors 116 and cleaning members 118 may be provided along with three larger diameter end effectors 116 and cleaning members 118.

Cleaning device 10/110 may be used to clean, for example, medical instruments, including metal (e.g., stainless steel, titanium, gold, copper, and aluminum), plastic, rubber, or silicone instruments, including, but not limited to, endoscopes, probes, cannulas, bed pans, stands, instrument trays, etc. During cleaning, cleaning device 10/110 may be used while either partially or completely submerged in liquid, while intermittently exposed to liquid (e.g., sprays or streams), or in dry conditions.

For cleaning device 10, a user may determine a suitable angle of deflection for end effector 16, which may either include an angle deflected from axis 24 or may include the neutral, on-axis configuration. The desired angle may be determined, at least in part, on the type of device to be cleaned, the shape and size of the device, the portion of the device to be cleaned, the dirtiness of the device, the position of the user relative to the device during cleaning, or the size of the user, for example. The device may be articulated to achieve the desired angle of deflection.

For embodiments in which cleaning member 18/118 is detachable, the user may attach a desired cleaning member 18/118. In embodiments for which a number of different cleaning members 18/118 are included, a user may first select a suitable cleaning member 18/118 from among the different types. This selection may be based, at least in part, on the type of instrument to be cleaned, the material of the cleaning member and the material of the medical instrument, the shape and size of the instrument, the portion of the instrument to be cleaned, the dirtiness of the instrument, the position of the user relative to the instrument during cleaning, or the size of the user, for example. In some embodiments, the selection may be predetermined based on a prescribed procedure for cleaning the medical instrument.

Once cleaning member 18/118 is in place and end effector 16/116 is positioned, a user may contact cleaning member 18/118 to the medical instrument for cleaning. During the cleaning process, the user may grip base member 12/112 with either one or two hands and may contact cleaning device 10/110 against the medical instrument. If cleaning device 10 being used includes light source 22 on end effector 16, the user may turn on light source 22 either before, during, or after the cleaning process to view the cleaning area. If cleaning device 10/110 includes a moveable end effector 16/116 and/or cleaning member 18/118, the user may also initiate movement of cleaning member 18/118 either before or during the cleaning process to facilitate cleaning. In some embodiments, the same actuator 30 may both turn on light source 22 and initiate movement, and thus the user may accomplish both at once. Depending on the embodiment, the user may select a speed of movement, select a type of movement, select a type of light, or select a brightness level, or may adjust any of these, before, during and/or after the cleaning process. Additionally, in embodiments having a detachable cleaning member 18/118, the user may detach and replace cleaning member 18/118 before, during or after the cleaning process, for example, to replace a damaged or dirty cleaning member or to switch the type of cleaning member used.

During cleaning, one or more cleaning products may be applied to a portion of cleaning device 10/110. As discussed above, cleaning device 10/110 may be submerged in a liquid, e.g., water, alcohol, hydrogen peroxide, bleach, soap, and/or any suitable cleaning fluid and/or combination of fluids during any portion of the cleaning process. Cleaning device 10/110 may either be partially submerged or completely submerged in such liquids, and may include one or more waterproof seals or coatings, or coatings to protect cleaning device 10/110 from acidic or basic liquids, such as industrial-strength cleaners, as described above.

A method of using cleaning device 10 to clean a medical instrument may include configuring articulation unit 14 to achieve a desired angle of deflection from axis 24. The angle of deflection may include a neutral, on-axis configuration, or may include a deflected, off-axis configuration.

For all embodiments of cleaning device 10/110, the user may submerge the medical instrument in a fluid and, grasping base member 12/112, may submerge at least a portion of cleaning device 10/110 in the fluid. The user may actuate movement of cleaning member 18/118 and/or may actuate lighting of light source 22 if cleaning member 10/110 has a light source. The user may contact cleaning member 18/118 to the medical instrument. The user may also agitate the fluid using the moving cleaning member 18/118. Further, the user may add one or more fluids to the liquid in order to facilitate cleaning. The user may adjust the motion of cleaning member 18/118 and/or may adjust light source 22. The user may illuminate and/or inspect the surface of the medical instrument to determine whether the instrument is clean. In embodiments with detachable cleaning members 18/118, the method may also include selecting a cleaning member 18/118 and attaching a cleaning member 18/118 prior to submerging cleaning device 10/110. Additionally, the detachable cleaning member 18/118 may also be removed and replaced during the cleaning process. For example, a cleaning member 18/118 may be replaced when it becomes worn out or damaged. A cleaning member may be worn out, for example, when the bristles or other components become degraded beyond a predetermined threshold. Alternatively, a cleaning member becoming worn out may be determined based on length of use. For example, after so many minutes, hours, or days of use the cleaning member may be deemed worn out. The steps of this cleaning process may be performed in any suitable order, and any of the steps may be repeated as deemed fit.

In some embodiments, during the cleaning of the medical instrument cleaning device 10/110 may be removed from the liquid and battery 34/134 may be removed and a replacement battery may be installed. For example, when battery 34/134 gets lower or below a threshold level, replacement of battery 34/134 may be desired. In some embodiments, during the cleaning the battery 34/134 removed from the cleaning device may be recharged by plugging in charger 139.

In some embodiments, during the cleaning process, the alignment of cleaning member 18 along axis 24 of end effector 16 may facilitate cleaning by providing the user with the ability to more accurately control and manipulate the desired angle via manipulation of articulation unit 14. Further, in embodiments that include light source 22 on end effector 16, the alignment of light source 22 with cleaning member 18 to provide directed lighting may also facilitate cleaning by illuminating the region where cleaning device 10 contacts the medical instrument, providing better visualization. Because end effector 16 may be deflected as a unit, relative to base member 12, directed illumination of a cleaning surface in the direction of cleaning member 18 may be achieved at any deflection angle, allowing more accurate control of illumination and cleaning angles.

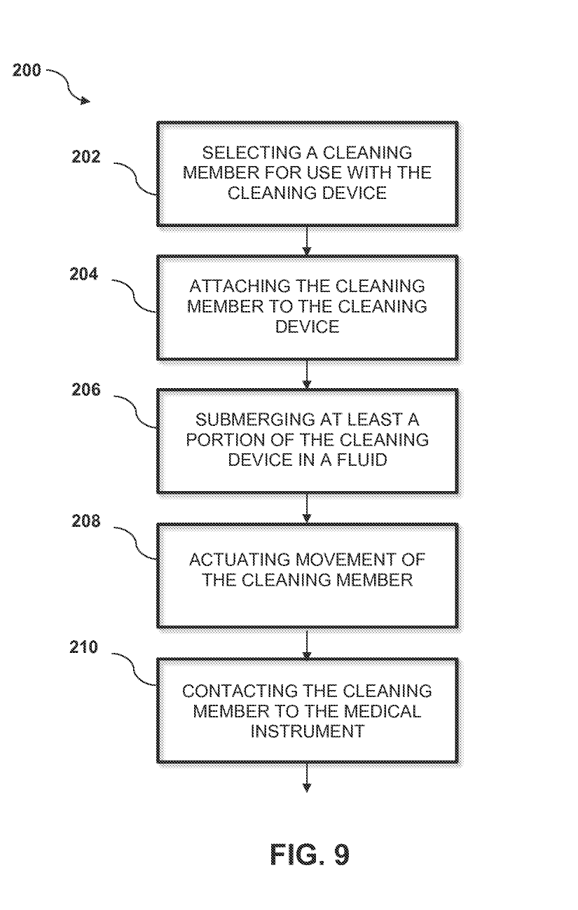

A method 200 of cleaning a medical instrument using a cleaning device, for example, cleaning device 10/110 is illustrated by the flow chart of FIG. 9. Method 200 may comprise steps 202, 204, 206, 208, and 210. Step 202 may comprise selecting cleaning member 18/118 for use with cleaning device 10/110. Step 204 may comprise attaching cleaning member 18/118 to cleaning device 10/110. Step 206 may comprise submerging at least a portion of cleaning device 10/110 in a fluid. Step 208 may comprise actuating movement of cleaning device 10/100. Step 210 may comprise contacting cleaning member 18/118 to the medical instrument. Method 200 may include additional steps including repeating one or more of steps 202, 204, 206, 208, and 210. In addition, method 200 may include additional different steps including the various steps described above with regard to cleaning devices 10/110.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

What is claimed is:

1. A method of cleaning a medical instrument using a cleaning device, comprising:
selecting a cleaning member for use with the cleaning device;
attaching the cleaning member to the cleaning device;
submerging at least a portion of the cleaning device in a fluid;
actuating movement of the cleaning member; and
contacting the cleaning member to the medical instrument.

2. The method of claim 1, wherein movement of the cleaning member includes at least one of rotation, vibration, translation, oscillation, and reciprocation.

3. The method of claim 1, further comprising agitating the liquid with the movement of the cleaning member.

4. The method of claim 1, further comprising altering the movement of the cleaning member.

5. The method of claim 1, wherein selection of the cleaning member is based upon the medical instrument being cleaned.

6. The method of claim 5, wherein selection of the cleaning member is predetermined based on a procedure for cleaning the medical instrument.

7. The method of claim 1, wherein selection of the cleaning member is based on the dirtiness of the medical instrument being cleaned.

8. The method of claim 1, wherein selection of the cleaning member is based on a material of the cleaning member and a material of the medical instrument.

9. The method of claim 1, further comprising replacing the cleaning member during the cleaning when it becomes worn out or damaged.

10. The method of claim 1, further comprising submerging the entire cleaning device in the fluid.

11. The method of claim 1, further comprising removing the cleaning device from the liquid and removing a battery from the cleaning device and replacing with a spare battery when the battery is low.

12. The method of claim 11, further comprising recharging the battery removed from the cleaning device by plugging a charger cord into the battery.

13. The method of claim 1, further comprising actuating illumination of a light source of the cleaning device.

14. The method of claim 1, further comprising configuring an articulation unit of the cleaning device to achieve a desired angle of deflection for an end effector of the cleaning device.

15. The method of claim 14, wherein the cleaning member is detachably connected to the end effector.

16. The method of claim 1, wherein the cleaning member includes at least one of bristles, protrusions, and textured elements.

17. The method of claim 1, wherein the cleaning device is part of a kit that includes the cleaning device, a plurality of cleaning members, a spare battery, and a charger cord.

18. The method of claim 17, wherein the kit is specifically designed for cleaning the medical instrument.

19. The method of claim 1, wherein the cleaning member is one of a bristled cleaning member, a textured cleaning member, a projection cleaning member, or a flexible cleaning member.

20. A method of cleaning a medical instrument, comprising:
configuring an articulation unit of a cleaning device to achieve a desired angle of deflection for an end effector;
selecting a cleaning member to attach to the end effector;
attaching the cleaning member to the end effector;
submerging at least a portion of the cleaning device in a fluid;
actuating movement of the cleaning member;
actuating illumination of a light source on the end effector; and
contacting the cleaning member to the medical instrument.
